(19) United States
(12) Patent Application Publication
(10) Pub. No.: US 2011/0118556 A1
(43) Pub. Date: May 19, 2011

(54) Systems and Methods for Diagnostics, Control and Treatment of Neurological Functions and Disorders by Exposure to Electromagnetic Waves

(76) Inventors: Peter H. Siegel, La Canada, CA (US); Victor Pikov, Pasadena, CA (US)

(21) Appl. No.: 12/945,553

(22) Filed: Nov. 12, 2010

Related U.S. Application Data
(60) Provisional application No. 61/261,190, filed on Nov. 13, 2009.

(51) Int. Cl. A61B 5/00; A61N 5/00; A61M 37/00

(52) U.S. Cl. 600/300; 600/1; 604/500

(57) ABSTRACT

Methods, systems and devices are provided in which electromagnetic waves (EMWs) are applied to a neural target. The neuronal function of at least a portion of the neural target is monitored, and the effect of EMW application on neural properties, neural function, and/or neural disorders is assessed. Embodiments include methods of diagnosis, treatment, drug delivery, cognitive enhancement, and EMW stimulation systems.

### CROSS-REFERENCE TO RELATED APPLICATIONS

This application claims the benefit of Provisional Patent Application No. 61/261,190, filed on Nov. 13, 2009, which is incorporated by reference herein.

### STATEMENT REGARDING FEDERALLY SPONSORED RESEARCH OR DEVELOPMENT

The invention described herein was made in the performance of work under a NASA contract, and is subject to the provisions of Public Law 96-517 (35 USC 202) in which the Contractor has elected to retain title.

### BACKGROUND

1. Field of the Invention

The invention relates to a system and method for exposure of neural cells to electromagnetic waves.

2. Related Art

Neurostimulation (including neuromodulation) involves the altering or modulating of neuronal activity by applying electrical, electromagnetic, or chemical agents directly to a target area. For example, brain stimulation has been applied for treatment of movement disorders {1} and neurodegenerative disorders {2}, for epilepsy {3}, and for chronic pain relief {4}. Similarly, spinal cord stimulation was used to relieve chronic pain {5}. Peripheral nerve stimulation was used for treatment of pain {6}, eating disorders {7}, and neuropsychiatric disorders {8}. In these techniques, electrical stimulation has been used to stimulate neuronal structures in specific areas of the nervous system to either excite or inhibit neuronal activity that is associated with a particular neurological disorder. Electrical stimulation typically uses invasive electrodes for delivery of electrical energy into neural tissue.

Increasingly high-frequency electromagnetic waves (EMWs) in the range of 3-300 GHz are being employed for telecommunication, radar, security, and imaging applications {9-11}. However, very few investigations on the impact of this radiation on biological systems at the cellular level have been undertaken.

### SUMMARY

It has been found that EMWs can alter neuronal properties and patterns of neuronal activity {12; 13}. The current application describes methods and systems for EMW stimulation of neural cells and tissues.

In one aspect, a method of diagnosing a neural disorder is provided. The method includes: applying EMWs at a frequency in the range of 3 to 300 GHz to a neural target of a subject in an amount sufficient to alter a neural property of the target; comparing the neural property of the target after EMW application to the same neural property of a similarly treated control target; and diagnosing a neural disorder in the subject when a difference in the neural properties is observed. The method can further include discontinuing the EMW application in the event that the EMWs are applied in a manner that exceeds a predetermined upper EMW application limit or evokes undesired physiological effect(s). In any embodiment of the method, the applying of the EMWs can include applying several EMW amplitudes, base frequencies, or modulation frequencies, or applying EMWs in a duty cycled manner. The neural target in any embodiment can be the cerebral cortex, deep brain, brainstem, spinal cord, optic tract, optic nerve, retina, cranial nerves, spinal roots, peripheral nerves, or nerve endings in the skin, or a combination thereof.

In another aspect, a method of treating, halting deterioration of, or preventing a neural disorder is provided. The method includes: exposing a neural target of a subject in need of such treatment to EMWs at a frequency in the range of 3 to 300 GHz in an amount sufficient to alter a neural property of the neural target, thereby conferring a therapeutic benefit on the subject, where the neural target is identified with a neural disorder. The method can further include discontinuing the EMW exposure in the event that the EMWs are applied in a manner that exceeds a predetermined upper EMW exposure limit or evokes undesired physiological effect(s). In any embodiment of the method, the exposing of the neural target includes exposing the neural target to several EMW amplitudes, base frequencies, or modulation frequencies, or to EMWs in a duty cycled manner. The method in any embodiment can further include monitoring neuronal function of the subject. In the method, the neural target can be the cerebral cortex, deep brain, brainstem, spinal cord, optic tract, optic nerve, retina, cranial nerves, spinal roots, peripheral nerves, or nerve endings in the skin, or a combination thereof.

In a further aspect, a method of delivering a drug is provided. The method includes: exposing a neural target in a subject's central nervous system (for example, cortex or spinal cord) or optic system (for example, retina) to EMWs at a frequency in the range of 3 to 300 GHz in an amount sufficient to alter the subject's blood-brain or blood-retinal barrier, respectively; and administering a drug to the subject before, during, or after the EMW exposure, or any combination thereof, such that the drug crosses the blood-brain or blood-retinal barrier. In this embodiment, exposure to EMWs can result in an increase in the amount of drug crossing the blood-brain or blood-retinal barrier compared to the subject before EMW exposure, or compared to a control subject. Examples of drugs include, but are not limited to, the following categories: neuroprotective (against hypoxia and excitotoxicity), neurotrophic, anti-ischemic, anti-apoptotic, anti-inflammatory, against oxidative stress, promoting re-sealing of the blood-brain or blood-retinal barrier, vasodilating, vasoconstrictive, anti-epileptogenic, anticonvulsive, anti-amnesic, analgesic, sedative, anti-neurodegenerative, anti-tremor, anti-parkinsonian, anti-psychotic, mood stabilizing, anxiolytic, and anti-depressant, and a combination thereof. Examples of drugs include, but are not limited to: glutamate release inhibitors, glutamate receptor antagonists, monoamine oxidase inhibitors, Ca²⁺ channel blockers, GABA receptor agonists, gangliosides, neuroptophic factors, calpain inhibitors, caspase inhibitors, free radical scavengers, and steroids, or a combination thereof.

Another aspect is a method of cognitive enhancement. The method includes exposing a neural target in a subject's central nervous system to EMWs at a frequency in the range of 3 to 300 GHz in an amount sufficient to alter a neural property of the neural target, thereby increasing cognitive ability in the subject.

A further aspect is an EMW stimulation system for applying EMWs to a subject. The system includes: a housing that is implantable, portable, head-mounted, skin-mounted, or eye-mounted, or a combination thereof; a control unit, for controlling production of EMWs; at least one EMW source carried by the housing and operatively coupled to the control unit; and an EMW delivery device comprising at least one emission site operatively coupled to the EMW source. In the system, the housing can be constructed from a flexible polymer substrate upon which the wave delivery device is positioned, where the wave delivery device includes metal traces for forming a surface waveguide and an output EMW shaping element coupled to the waveguide. Any embodiment of the system can further include a unit for monitoring neuronal function operatively coupled to the control unit.

In some embodiments of the system, the EMW delivery device includes an elongate member and first and second EMW emission sites carried by the elongate member, where the first and second emission sites are separated along a length of the elongate member by a predetermined distance. In these embodiments, the predetermined distance can correspond to an expected approximate distance between two adjacent neural targets. In some embodiments of the system, the EMW delivery device comprises: an elongate member that includes a proximal segment and a distal segment, where the segments have different geometric shapes; and a first wave emission site carried by the elongate member. The distal segment in some embodiments has a geometric shape corresponding to a boundary of an anatomical structure and/or producing a predetermined exposure region on the anatomical structure.

### BRIEF DESCRIPTION OF THE DRAWINGS

For a more complete understanding of the present invention, reference is now made to the following descriptions taken in conjunction with the accompanying drawings.

FIG. 1A is a drawing showing an embodiment of an EMW stimulation system.

FIG. 1B is a drawing showing various embodiments of EMW delivery devices.

FIG. 2 is a drawing showing an embodiment of an EMW stimulating system with multiple sources.

FIG. 3 is a panel of sample recordings of neuronal activity in neuron n14 selected to show four incident power density (IPD) levels of EMW exposure, applied in a random order during the experiment. For each record, two traces are shown: the top trace shows the intracellular voltage and the bottom shows the injected current. Bars indicate the duration of the EMW exposure with the value above the bar indicating the IPD level.

FIG. 4 is a panel of graphs showing the effect of the IPD on the firing rate (A) and action potential (AP) amplitude (B) in eight neurons.

FIG. 5 is a panel showing sample AP profiles in neuron n14 before EMW exposure and at 50 s since the beginning of exposure at different IPD levels (indicated above each profile). In the experiment, the IPDs were applied in a random order.

FIG. 6 is a panel of graphs showing the effect of the IPD on the τᵣᵢₛₑ (A) and τ_{decay} (B) in eight neurons.

FIG. 7 is a graph showing the effect of the IPD on the Rₙ in eight neurons.

FIG. 8 is panel of sample recordings showing inhibition of AP firing rate in a Retzius cell of the medicinal leech somatic ganglion at 3 EMW power levels (power at the waveguide output). EMW was applied for 60 seconds then shut off. Note rapid return to normal AP rate.

### DETAILED DESCRIPTION

In accordance with embodiments of the present invention, EMWs are applied to a neural target, or a neural target is exposed to EMWs. The neuronal function of at least a portion of the neural target can then be monitored, and the effect of EMW application on neural properties, neural behavior, and/or neural disorders can be assessed. The EMWs include electromagnetic waves in the microwave and/or millimeter wave range of the EMW spectrum.

Examples of a neural target include, but are not limited to, cerebral cortex (including different regions such as orbitofrontal cortex, prefrontal cortex, parietal lobe, sensory cortex, motor cortex, visual cortex, cingulate gyrus, Brodmann area 9, Brodmann area 10, Brodmann area 25, or Brodmann area 46, or a combination thereof), deep brain, brainstem (including different regions such as midbrain, pons, or medulla, or a combination thereof), spinal cord (including different regions such as cervical, thoracic, lumbar, or sacral, or a combination thereof), the optic tract, the optic nerve, retina, cranial nerves (including different types such as cochlear nerve, or facial nerve, or a combination thereof), spinal roots, peripheral nerves (including different types such as vagal nerve, pudendal nerve, pelvic nerve, or a combination thereof), and nerve endings in the skin, and a combination thereof.

In any embodiment, the EMWs: can be applied in the range from about 3 to about 300 GHz, either fixed or tuneable; can be applied as continuous wavelength (CW) or pulsed; can be modulated from 0 to 100 MHz; and can have an incident power density of within specified MPE (maximal permissible exposure) of 1 mW/cm² {14}; or a combination thereof; can be applied until they exceed a predetermined upper EMW application limit, such as the specific absorption rate of 1 W/kg; can be applied as several amplitudes; can be applied in a duty cycled manner, for example, by interrupting EMW application or exposure of the neural target for a predetermined time interval, and resuming the application or exposure after the predetermined time interval, based on the readout from sensors of neuronal properties or other closed-loop feedback controls. The operating parameters of the EMW stimulation system can be set according to these characteristics of the EMWs.

Examples of neural properties include, but are not limited to, cellular properties such as membrane potential, action potential shape, neurotransmitter release or uptake; and neuronal firing rate; tissue properties such brain wave formation; and nervous system properties such as behavioral responses, motor responses, sensory responses (such as visual responses, auditory responses), cognitive responses, autonomic responses, corticospinal conduction, sensory activation threshold, motor activation threshold, or degree of neurological impairment (such as the tremor severity, frequency of seizures, amount of spasticity).

Examples of neural disorders include, but are not limited to, seizure disorders (for example, epilepsy); movement disorders (for example, dystonia); hereditary and degenerative diseases of the central nervous system (for example, Parkinson's disease, essential tremor, Alzheimer's Disease, Huntington's chorea, hydrocephalus); pain (for example, chronic pain, central pain, peripheral pain); headache syndromes (for example, cluster headaches, tension type headache, post-traumatic headache, migraine); auditory dysfunctions (for example, tinnitus, hyperacousis, hearing loss); visual dysfunctions; stroke; coma, vegetative state, or minimally-conscious state; cognitive/memory dysfunction; traumatic brain injury, blast injury, or spinal cord injury; demyelinating diseases (for example, multiple sclerosis); anterior horn cell diseases (for example, amyotrophic lateral sclerosis, locked-in syndrome); disorders of the autonomic nervous system; cerebral palsy; spinocerebellar diseases (for example, spastic paraplegia); inflammatory diseases of the central nervous system (for example, meningitis, encephalitis, myelitis, and encephalomyelitis); psychiatric disorders (for example, depression, schizophrenia, addition, anxiety disorders, panic disorder, obsessive-compulsive disorder, attention-deficit hyperactivity disorder, bipolar disorder, alcohol and narcotics dependence, post-traumatic stress disorder). Also, disorders such as detached retina or recurrent glioblastoma can be treated with EMW therapy, for example, by photothermal adhesion of detached retinas or heating of the brain tumor.

In diagnostic embodiments, EMWs are applied to a neural target of a subject, and a neural property of the irradiated target is compared to the same neural property or a corresponding neural property of a similarly irradiated control target (typically, a normal target). A difference in the neural properties indicates that the subject has a neurological disorder, neural trauma, or other changes prognostic or precursory of neurological disorder(s). The particular neural target irradiated and the particular neural property compared will depend on the particular disorder to be diagnosed. For example, when diagnosing traumatic brain injury, the neural target is the brain and the diagnostic neural properties are the pattern of neuronal activity in the cortex and amount of blood and extracellular fluid in the brain tissue indicating the development of hematoma and edema; or when diagnosing retinal disorders such as retinitis pigmentosa, age-related macular degeneration, or other retinal diseases, the neural target is the retina and the diagnostic neural property is the excitability of photoreceptor neurons in the retinal pigment epithelium layer.

In treatment embodiments, a neural target in a subject is exposed to EMWs in an amount sufficient to alter a neural property of the target. This confers a therapeutic benefit on the subject. The term “therapeutic benefit” used throughout this application refers to anything that promotes or enhances the well-being of the subject with respect to the medical treatment of the subject's condition, which includes treatment of neural disorders. A list of nonexhaustive examples of benefits includes extension of the subject's life by any period of time, a decrease in pain to the subject that can be attributed to the subject's condition, a decrease in the severity of the disease, an increase in the therapeutic effect of a therapeutic agent, an improvement in the prognosis of the condition or disease, a decrease in the amount or frequency of administration of a therapeutic agent, an alteration in the treatment regimen of the subject that reduces invasiveness of treatment, and a decrease in the severity or frequency of side effects from a therapeutic agent. In these embodiments, the particular neural target is one that is identified with, or associated with, a particular neural disorder. For example, the neural targets for seizure disorders are the cerebral cortex, basal ganglia, brainstem, cerebellum, and thalamus; the neural targets for Alzheimer's disease are the cerebral cortex (specifically the frontal, temporal, and parietal regions), hippocampus, amygdale, and cingulate gyrus; the neural target for dementia is the neocotex; the neural targets for Parkinson's disease are the cerebral cortex, basal ganglia, hypothalamus, brainstem, and spinal cord; the neural targets for chronic pain are the spinal cord, brainstem, spinal roots, peripheral nerves and cranial nerves; and the neural targets for peripheral pain are the peripheral nerves and nerve endings in the skin.

In cognitive enhancement embodiments, examples of cognitive ability that can be increased or altered include, but are not limited to, the ability to store, retain, and/or recall (in short-term and long-term) the sensory (visual, auditory), verbal, spatial, kinesthetic, logical, musical, procedural, and/or declarative memories. Particular measurements of cognitive ability will depend on the particular ability to be enhanced. For example, the ability to store and retain visual memories can be measured by presenting a set of images for memorization followed by a delayed presentation of a test set of images containing some images from the first set and some new images.

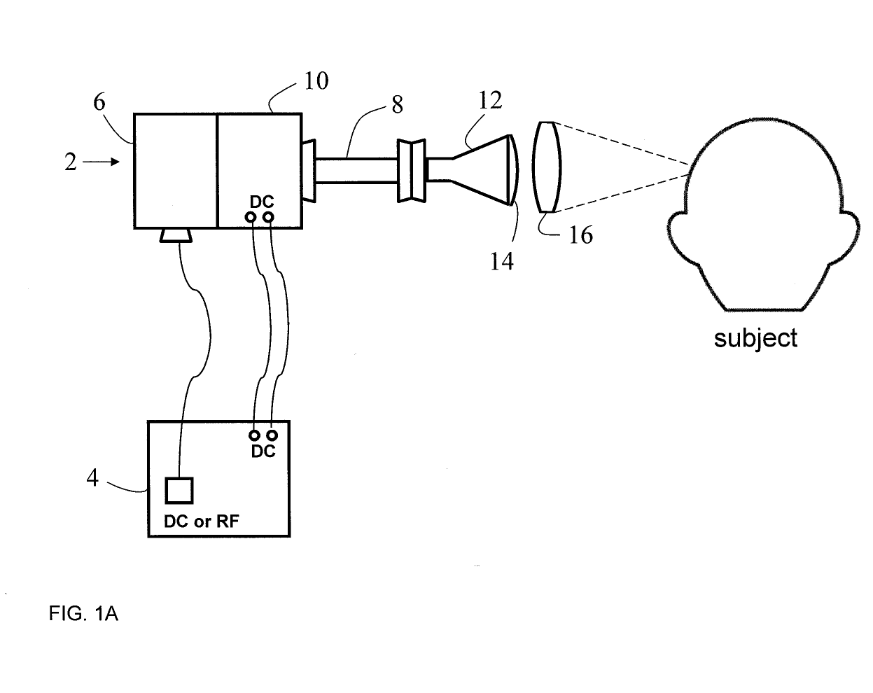

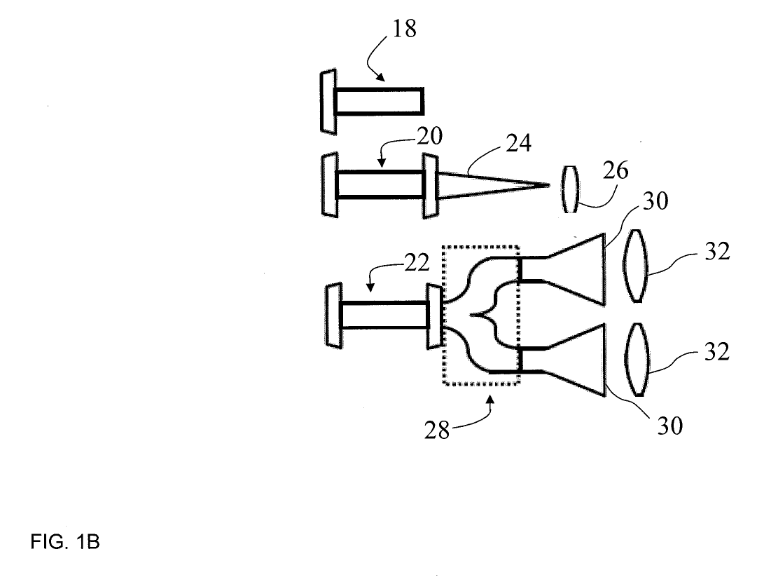

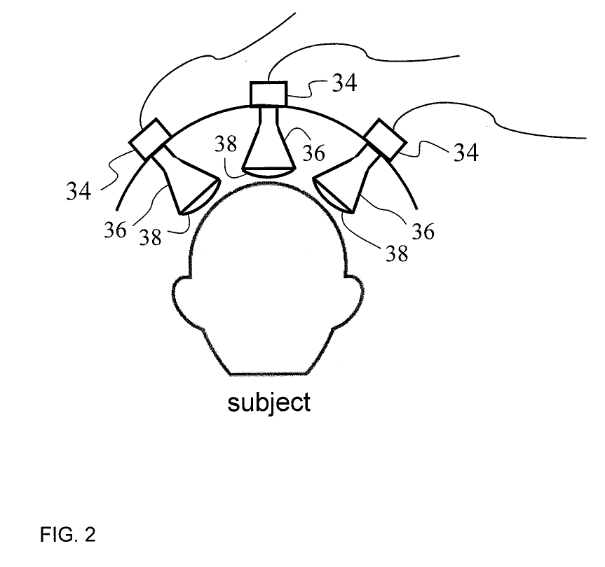

Referring to FIG. 1A, in embodiments including an EMW stimulation system, the system can include the following components: a housing 2 that is implantable, portable, head-mountable, skin-mountable, or eye-mountable, or a combination thereof; a control unit 4, for controlling production of EMWs, for power supply, and/or for telemetry; at least one EMW source 6 carried by the housing and operatively coupled to the control unit; and an EMW delivery device 8 comprising at least one emission site operatively coupled to the EMW source; and an optional amplifier 10. A portable housing is capable of being connected to a subject for personal use. A head-mountable, skin-mountable or eye-mountable housing is able to be used on or in the head, skin or eye, respectively, under normal operating conditions. In FIGS. 1A and 2, the housing is coupled to the EMW delivery device, but in other embodiments the EMW delivery device can be separated from the housing.

In some embodiments, the system or portion of the system is used for direct-contact placement, that is, placement on or in a tissue or organ. EMWs can then be applied directly to a neural target in the tissue or organ. Examples of direct-contact placement include, but are not limited to, epidurally such as on the cerebral cortex, brainstem, or spinal cord; epiretinally; subretinally; suprachoroidally; intravascularly; or intraventricularly. In some embodiments, the system is used for superficial placement, on the skin or other body surface. EMWs can then be applied directly to a neural target on or in the skin or other body surface, or indirectly through the skin or other body surface to a neural target below. Examples of superficial placement include, but are not limited to, on the scalp such as for transcranial brain irradiation; on the skin above the brainstem, spinal cord or peripheral nerve; epicorneally (for example, like a contact lens); and mounted like glasses in front of the eye.

For direct-contact placement, an implantable housing can be made from flexible polymer substrate (for example, polyimide, LCP, polytetrafluoroethylene, SU-8 photoresist, polyethylene, polypropylene, TPX, polystyrene, or parylene) and patterned with metal traces for surface waveguides and beam shaping elements such as horns and lenses or mirrors. The substrate can be manufactured using different metal machining or lithographic technologies known in the art, such as: nanoimprint lithography (either thermoplastic/hot embossing or photo/UV-based), optical lithography, electron beam lithography, X-ray lithography, or synchrotron radiation etching. Metal based features, such as the circuits and waveguides, can be attached to the substrates or patterned directly on the substrate using different microfabrication procedures known in the art, such as: electroplating, liftoff, spinning, plasma etching, sputter deposition. The parallel nature of these procedures allows patterning of multiple features on the same substrate at the same time. Single or multiple layers of polymer substrates and metal patterns are used in combination to create the waveguides, antennas, and horns for shaping and delivery of EMWs. These waveguides and horns can be either planar or three-dimensional in structure. They can take the form of conical, pyramidal or ellipsoidal horns, convex and concave lenses or mirrors, open ended or dielectrically loaded waveguides, wire or multi-electrode antennas, or shaped dielectric antennas. The EMW emitter can be externally applied to the target neural area (such as through the skin) or can be injected through a catheter, endoscope or directly through intervening bone or thin tissue layers to the neural tissue using both TEM coaxial waveguides, dielectric guides, also circular, rectangular or other common geometrically shaped guides which can carry the dominant mode. For superficial placement, the system can contain an EMW delivery component coupled to a beam shaping component. Examples of some common waveguides and beam shaping elements are shown in FIGS. 1 and 2, but others known in the art also can be used.

The EMW source can be a device known in the art such as a backward-wave oscillator, orotron, Gunn diode oscillator, IMPATT Diode, Solid State Gunn Diode, synthesizer and upconverter, oscillator and discrete or MMIC amplifier, YIG tuned oscillator, dielectric resonator, vacuum tube oscillator, clinotron, gyro-klystron, gyrotron, traveling wave tube, gyro-traveling wave tube, or pulsed magnetron. In some embodiments, the control unit can be housed together with a power supply for providing power to, for example, the EMW source and an amplifier. In other embodiments, the power supply can be housed separately from the control unit. Although the control unit in FIG. 1A is placed in a separate housing from the EMW source, the control unit can be place in the same housing as the EMW source in other embodiments. The EMW delivery device can be a device known in the art such as a planar or 3D hollow or dielectric waveguide, direct source to antenna coupled radiators, planar antenna coupled oscillator and amplifier, wire or planar rectenna or any known source-coupled RF radiator. The delivery device can be single or multimode, rectangular, round, square, ridge, elliptical, or the like. Referring to FIG. 1B showing some embodiments, the delivery device can be an open ended device 18, a dielectrically loaded device 20, or a power divided device 22.

Referring to FIGS. 1A and 1B, one or a combination of additional components in any embodiment can include an amplifier 10 (such as transistor amplifiers, MMIC amplifiers, tube amplifiers, hybrid amplifiers, and other solid state amplifiers), and elements 12 for shaping the EMW output beam. The EMW beam shaping element can be an optical element (for example a lens or a mirror, optionally movable for depth and lateral focusing) or an antenna (for example a linear, planar circular, planar horn, or 3D horn antenna). For example, a rod antenna 24 and lens 26 for beam shaping are shown in FIG. 1B. Other components include: a collimating element 14 such as a dielectric lens, or a spherical, parabolic, elliptical or conical mirror; and a focusing element 16 such as a lens or curved mirror. The various components of the system can be arranged as single or multiple sources, and can be organized in 1D and/or 2D arrays. For example, FIG. 1B shows an embodiment of a splitter or power divider 28 for forming a liner or 2D array of transmitters with individual beam forming elements 30 and focusing elements 32, such as lenses or mirrors. Also, FIG. 2 shows a multiple-source stimulation setup that includes EMW sources 34, beam forming horns 36, and a focusing device 38 in the form of an integrated lens. The setup can be arranged as a 1D or 2D array of devices distributed on a plane or contoured surface to illuminate multiple regions of the subject.

As an added feature of the system, method or any other embodiment, the neuronal function of a neural target or other neural region can be monitored. For example, in any embodiment, neuronal activity and structure can be monitored through one or a combination of the following detector types and procedures: electroencephalography (EEG); magnetoencephalography (MEG); optical or near-infrared imaging/spectroscopy; Doppler blood flow monitoring; computer tomography (CT); single photon emission computed tomography (SPECT); magnetic resonance imaging/spectroscopy systems (MRI/MRS); sensing of RF conductivity and permittivity sensing of RF back scattering/forward scattering; and sensing of electrical impedance; or a combination thereof. In diagnostic embodiments, for example, diagnostic brain mapping/tomography can be performed by combining EMW irradiation with functional MRI (fMRI), EEG or MEG. A detector device is typically physically separate from other components of the system and can be hard-wired or wirelessly connected to one or more components of the system.

The system can also include: a capacity for detecting local temperature by RF or optical (visible, infrared) thermometry; a capability for detecting tissue pressure using piezoelectric or ultrasound sensors; a power supply provided through wires or wirelessly using radiofrequency (e.g. kHz, MHz, or GHz) inductive coupling, or infrared power telemetry; the capability to tune the applied power, frequency, modulation, and localization of applied irradiation; a telemetry circuit for wireless communication with an external controller/programming device; or a combination thereof. In addition, EMWs can be delivered either automatically through a pulse generator built into the stimulation system, or using commands from an external controller that can communicate with the stimulation system.

The system can include a safety protection feature that discontinues EMW stimulation in the event that the EMWs are applied in a manner that exceeds a safety limit. Safety limits can be defined based upon the output of the EMW source (such as the incident power density within specified MPE of 1 mW/cm² {14}, or the specific absorption rate within 1 W/kg) or upon the detection of physiological responses (body temperature, blood pressure rate, blood oxygenation, respiration, local neuronal activity) exceeding the normal limits.

In some cases, embodiments disclosed herein relate to systems, apparatus, devices, and methods for providing, applying, or delivering the EMW stimulation to neural targets within the brain, brainstem, spinal cord, eye, peripheral nerve, any region of the skin and/or elsewhere. More particularly, embodiments disclosed herein relate to irradiating neural targets in situations in which a neurostimulating, neuromodulatory, or neuroprotective effect may be desirable.

Some embodiments relate to systems and methods for treating neurological disorders by electromagnetic stimulation in the EMW range. According to aspects illustrated herein, there is provided an EMW delivery device that includes an elongate member; a first EMW emission site carried by the elongate member; and a second EMW emission site carried by the elongate member, wherein the first EMW emission site and the second EMW emission site are separated along a length of the elongate member by an expected approximate distance between two neural targets or a single EMW emitter with a beam shaped to target a defined region of the skin or other surface.

According to aspects illustrated herein, there is provided an EMW delivery device that includes an elongate member including a proximal segment and a distal segment, the proximal segment having a different geometric shape than the distal segment; and a first EMW emission site carried by the elongate member, wherein the distal segment has a geometric shape that corresponds to a boundary of an anatomical structure. In an embodiment, the EMW delivery device further includes a second EMW emission site carried by the elongate member.

According to aspects illustrated herein, there is provided a neuronal EMW stimulation system that includes an implantable housing; a control unit carried by the implantable housing; an EMW delivery device including an elongate portion and a contoured portion, the EMW delivery device having at least a first EMW emission site; and an EMW source operatively coupled to the control unit and coupled to the first EMW emission site, the EMW source carried by one from the group of the implantable housing and the EMW delivery device, wherein the contoured portion of the EMW delivery device has a geometric shape that corresponds to a boundary of an anatomical structure. In an embodiment, the neuronal EMW stimulation system further includes a unit for monitoring neuronal function coupled to the control unit, wherein the unit is configured to detect an electrical, optical, or magnetic signal corresponding to neuronal activity. In another embodiment, the neuronal EMW stimulation system further includes a telemetry circuit carried by the implantable housing; and a programming device configured for wireless signal communication with the telemetry unit.

According to aspects illustrated herein, there is provided a neural stimulation method that includes applying EMW stimulation to a neural target; and determining if the EMW stimulation is applied in a manner that exceeds a predetermined or programmable upper electrical stimulation limit. In an embodiment, the method further comprises determining if the electrical signals are applied in a manner that exceeds an expanded upper EMW stimulation limit. In an embodiment, the method further comprises discontinuing the EMW stimulation in the event that the EMW stimulation is applied in a manner that exceeds the upper EMW stimulation limit.

According to aspects illustrated herein, there is provided a method for treating a neural disorder of a patient that includes implanting an EMW delivery device into the patient and preferentially directing EMW stimulation toward a predetermined set of neural targets.

The use of the singular forms “a,” “an,” and “the,” both in the claims and the description, include plural references unless the context clearly dictates otherwise.

The present invention may be better understood by referring to the accompanying examples, which are intended for illustration purposes only and should not in any sense be construed as limiting the scope of the invention.

### Example 1

In beginning to examine the impact of EMWs on cellular processes {12; 13}, the inventors discovered that cell membrane potential and action potential firing in neurons may be altered by ultra low power levels at these short wavelengths. Such a situation could be used to advantage in the direct stimulation of neuronal cells for applications in neuroprosthetics and diagnosing or treating neurological disorders from epilepsy to peripheral pain. The inventors have found that low EMW power triggered membrane depolarization in vitro in epithelial cell, and without wishing to be bound by any theory, hypothesize RF induced membrane nanopore formation or opening of the voltage-sensitive channels and subsequent ion transport across the plasma membrane. In addition, through a series of in vitro patch clamp experiments using brain slices from a normal neonatal rat population, they recorded suppression and enhancement of neuronal firing rate, recoverable membrane depolarization, and narrowing of the action potential (AP) shape using 60 GHz CW radiation at power levels well below the recommended government safe limits (<<1 mW/cm²). In addition, action potential firing rate in individually probed Retzius cells from the neural ganglion of the medicinal leech showed recoverable inhibition and narrowing of the AP shape that scaled with increasing EMW power level. The inventors consider such control of nerve signaling to be an important therapeutic for pain management, neurological treatment and neuronal control via catheter or endoscopic means. Induced membrane depolarization in epithelial cells and neurons are also considered to have an important role in drug delivery and disease therapies.

### Cellular Responses

An experimental system was set up to directly monitor cell response on exposure to continuous wave fixed frequency EMW radiation at low and modest power levels (0.001-100× of safe exposure limit) between 50 and 100 GHz. Two immortalized cell lines derived from lung (epithelial) and neuronal tissue, were transfected with green fluorescent protein (GFP) that locates on the inside of the cell membrane lipid bilayer. Oxonol dye was added to the cell medium. When membrane depolarization occurs, the oxonol bound to the outer wall of the lipid bi-layer can penetrate close to the inner wall where the GFP resides. Under fluorescent excitation (488 nm) the normally green GFP (520 nm) optical signal quenches and gives rise to a red output when the oxonol comes close enough to the GFP to excite a fluorescence resonance energy transfer (FRET) with an output at 620 nm. The presence of a strong FRET signature upon exposures of 30 seconds to 2 minutes at 5-10 mW/cm² RF power at 50 GHz indicated membrane depolarization. This was followed by a return to the normal 520 nm GFP signal after a few minutes, indicating repolarization of the membrane. This experiment indicated that low levels of RF energy trigger non destructive membrane depolarization without direct cell contact. Such a mechanism could be used to stimulate neuronal cells in the cortex without the need for invasive electrodes as mm-waves penetrate skin and bone on the order of 1-10 mm in depth. Although 50 GHz could not readily penetrate from the outer skull to the depth of cerebral cortex, implants on the outer skull or even on the scalp could reach the outer layer of the cerebral cortex where substantial benefit could be realized from such non-contact type excitation. Furthermore, use of lower frequencies (3-30 GHz) would allow deeper penetration into the cerebral cortex. There would be a direct trade-off between signal localization and penetration depth based on excitation wavelength that could be used for deeper stimulation within the brain, brainstem, spinal cord, retina, or skin.

### Materials and Methods

Brain slices from two week old neonatal rats were incubated in a polycarbonate patch clamp dish (BT-1-18, Cell MicroControls) containing artificial cerebral spinal fluid (ACSF, NaCl, NaHCO3, glucose, all from Sigma-Aldrich, St. Louis, Mo.). Individual neurons were probed with pulled pipette containing an electrolytic solution (K-gluconate, KCL, NaCl, HEPES et. al., pH 7.2, recipe on request) and current sampling electrodes. Healthy cortical pyramidal neurons were evaluated by their resting potential (−70 mV typical) and their action potential firing rates (typically 5-10 spikes per second) which were continuously monitored and recorded on a Axopatch 700B amplifier and Digidata 1440 (Molecular Devices, Union City, Calif.) patch clamp system. The electrodes were pulled from recording glass pipettes (WPI, Sarasota, Fla.) with a P-1000 (Sutter Instruments) and had a resistance between 4 and 8 megaohms. The software used for control of stimulation and data recording is Clampex 10 (Molecular Devices). An applied stimulation current of 100 to 500 pA (200 pA typical) was used to stimulate neuron firing which typically occurred at −35 to −40 mV with peaks of 20-40 mV above these levels. The holding current was 0 pA. The stimulation was sequentially applied for 5 seconds, and then removed for 15 seconds (resting period) continuously over the course of many minutes under which the neuron was being monitored. During the elevated portions of the stimulus cycle the neurons fired repeatedly and continuously at varying rates until the stimulus was reduced. Threshold voltages for firing were typically Control measurements made by simply recording the normal firing rate for a series of stimulus and rest cycles, followed by application of the external RF signals. Visual indication of the cell field and patch clamp insert could be monitored on the upright microscope using an immersion objective before the RF signals were applied. However the objective was lifted out of position in order to perform the RF injection within a reasonable distance from the neurons. The applied EMW energy was sprayed over the entire brain slice using a single mode waveguide aperture elevated directly above and within a few millimeters of the fluid filled patch clamp dish. The EMW source was operated in continuous wave mode but able to be turned on and off by a mechanical rotary vane attenuator that continuously decreased the applied power without the need to throw any electronic switches during the measurements. The power levels at the output of the waveguide were measured to be 185 mW maximum and the inline attenuator allowed us to reduce this to any level while monitoring the incident power level through a calibrated directional coupler. The waveguide output port was elevated just to the edge of the far field distance of the aperture and was designed to have a wide beam spread (120 degrees H plane, 65 degrees E plane) so that the RF beam would uniformly illuminate the microscopic region under observation.

The neuronal current stimulus was set up in a repeating sequence of 5 seconds on, 15 seconds off with recordings collected continuously. The media flow rate was stopped and some of the fluid withdrawn from the dish (leaving 2.2 mm of fluid above the slice) during the RF exposures. RF power was applied during the off state and the impact on the subsequent on-state action potential firing rate could easily be compared to prior no-power sequences. At maximum applied power the firing rate could be reduced, fully suppressed and restored (by turning off the RF) depending on the length of time the neuron was probed. Direct temperature readings of the bath were made during the measurements and at no time was the temperature rise more than 3° C. even at the highest applied powers. The applied RF power level was reduced in steps until no changes could be observed between power-on and power-off states. This threshold level was between 22 and 33 mW at the waveguide output. Even at 33 mW however it was possible to fully suppress the action potentials.

The approximate RF power reaching the neurons is extremely hard to measure. The EMW exposure system consisted of a custom assembled EMW source (injection-locked IMPATT oscillator) operating at 60.125 GHz and producing up to 185 mW of continuous wave (CW) power at the exit of the open-ended WR-15 waveguide. Power was continually monitored through a directional coupler and calibrated RF power meter (HP 436A, Agilent Technologies, Santa Clara, Calif.). The power emitting on the tissue chamber was controlled by a rotary vane attenuator that could reduce the power to below detectable level (<1 μW) without throwing an electrical switching discharge. The rectangular waveguide with 3.8×1.9 mm aperture was carefully positioned above the tissue chamber, blocking the path of the microscope. The EMW power was directed perpendicular to the plane of the chamber for more uniform irradiation of the area occupied by the tissue slice. The tip of the waveguide was placed just beyond the far field distance of the tissue with an air gap of 4.8 mm to the surface of the ACSF solution and approximately 6 mm to the top of the slice. The linearly polarized RF fields exiting the rectangular waveguide expand at a half angle of ˜27° in the H plane (parallel to the wide wall) and 56° in the E plane (along the short wall) creating a half-power ellipse of 5.5 mm2 (major and minor axes of 14.2 and 4.9 mm) at the ACSF surface. The RF beam is refracted upon entering the solution and forms a half-power ellipse of 6.5 mm2 (major and minor axes of 15.2 and 5.4 mm) at the top of the slice, 2.2 mm below the ACSF surface. A large portion of the incident RF signal is reflected upon contact with the fluid surface and is assumed to be radiated into the surrounding space without absorption by the fluid. Using published data for the complex permittivity of water and water-based media at 60 GHz, we calculated the reflected power to be ˜45%, leaving 55% of available power for absorption by ACSF. Using the published data for the same frequency and similar ionic media and a Beer's law (I=I₀e^{-ax}) for the intensity drop with penetration depth x (in cm), we estimated the loss tangent for EMW absorption by ACSF to be 1.48. This translates into the absorption coefficient of 52 cm⁻¹, indicating the power drop of 99.5% by 1 mm of ACSF. Using a further approximation that the energy in the RF beam is uniformly distributed over the tissue within the half-power ellipse, the maximal power of 185 mW exiting the waveguide port produces a power density of approximately 90 mW/cm² transmitted into the ACSF and less than 1 μW/cm² at the tissue level 2.2 mm below the surface. Therefore, in this study, the highest applied incident power density (IPD) of EMWs at the level of slice neurons is 1000 times lower than the most conservative current safe exposure level of 1 mW/cm² {14}.

Whole-cell recordings were made in freshly-prepared slices of the cerebral cortex of neonatal rats. Neurons of a pyramidal phenotype located in the layer 2/3 of the cortex were selected using infrared videomicroscopy with DIC enhancement. A total of 8 neurons were patched in 8 slices from three rats. After the neuron was patched and its microscopic visualization was no longer necessary, the EMW exposure system was moved into place and used to illuminate the tissue chamber with a series of IPDs in a random order. Prior to the EMW exposure, the fluid flow through the chamber was halted and ACSF was partially removed from the well containing the tissue slice until visual confirmation of a concave meniscus formed at the fluid surface. The remaining amount of ACSF in the chamber was 0.9-1.1 ml and the depth of solution covering the slice was between 2.1 and 2.3 mm. This depth was sufficient to fully stabilize the temperature in the fluid above the slice and allow the sufficient nutrition to neurons during the measurement sequence lasting 30-60 minutes. A thermal sensor was located close to the slice, assuring that the slice was exposed to an equivalent amount of ACSF-damped heating from the EMW source. The actual power, generated by the EMW source, was measured throughout the experiment using an inline directional coupler and the calibrated power meter.

### Results

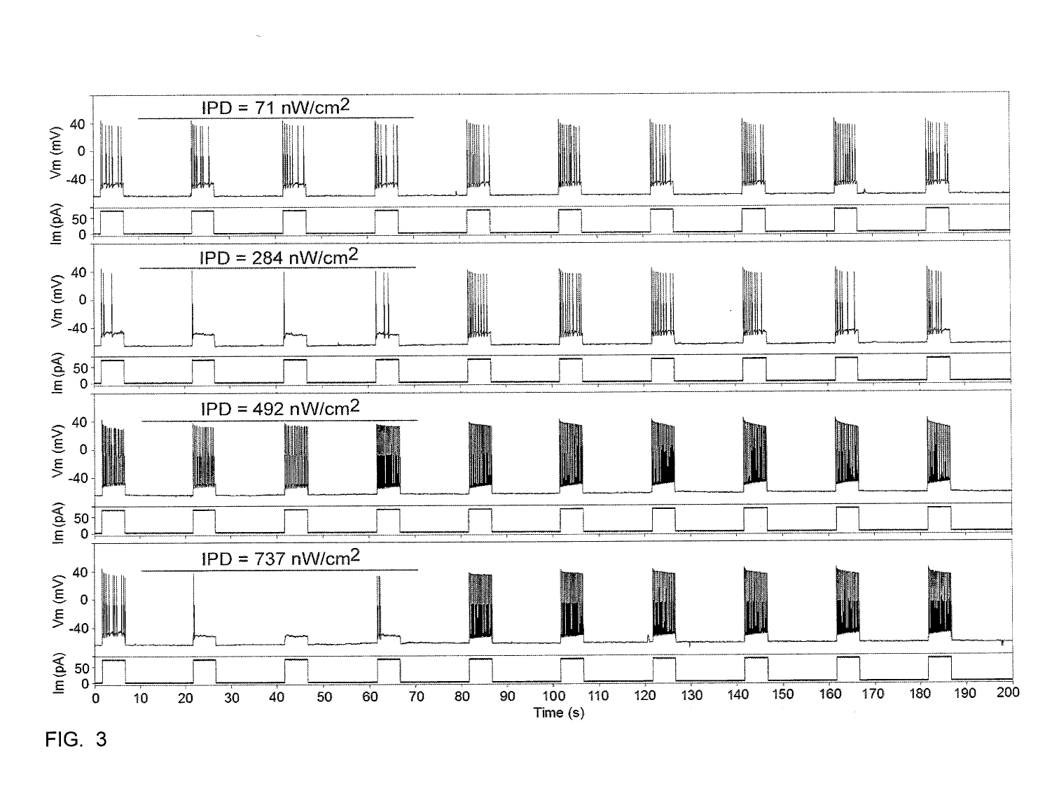

In order to improve neuronal viability and stability of neuronal firing in the whole-cell recordings, the depolarizing intracellular current was injected at a 25% duty (FIG. 3). Several EMW power levels were applied to the slice in a randomized order to remove any possible effects of cumulative exposure. Adequate time intervals (2-4 min) were used between the exposures to assure the recovery of baseline neuronal activity. Each exposure lasted for 60 sec (or three 20-sec cycles), thus three 5-sec neuronal activity records were collected during the exposure. In a presented example (FIG. 3), exposure of the neuron n14 with a low EMW IPD of 71 nW/cm² produced no significant effect on the neuronal firing rate during the exposure and a small increase in firing rate after the exposure. Higher IPD levels (from 284 to 737 nW/cm²) produced considerable inhibition of the neuronal firing during the exposure and strong facilitation of firing immediately after the exposure.

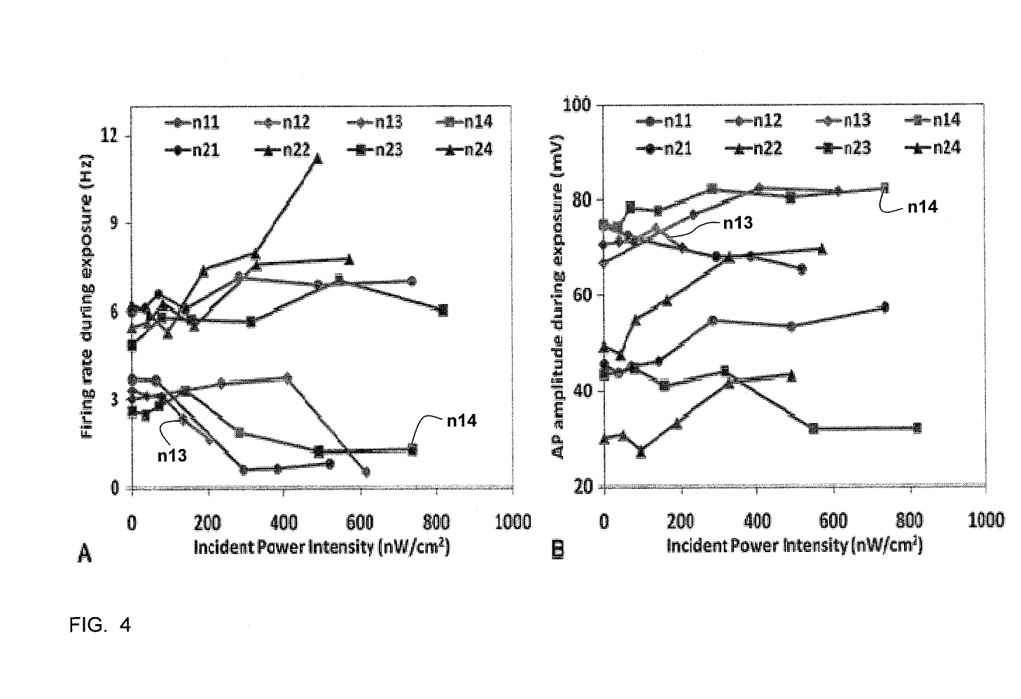

To evaluate the effects of EMW on firing rate and AP amplitude across several IDP levels, the average baseline value of the firing rate and AP amplitude was calculated. A ratio of the baseline value in the trial divided by the average baseline value was then used to normalize the values in the individual trials. This was necessary to reduce the variability among the trials, as can be seen in the trial with IDP of 492 nW/cm² in FIG. 3. No normalization was used to compare the effect of the EMWs on firing rate and AP amplitude among the neurons. Instead, it was decided to group the neurons based their baseline firing rate. Two groups were identified: first (n=4) with low (˜3 Hz) and second (n=4) with high (5-6 Hz) firing rate (FIG. 4 and Table 1). The low-firing group exhibited strong inhibition by the EMWs, while the neurons in the second group exhibited either facilitation (n=2) or remained relatively unchanged (FIG. 4A). The neurons in the first group also exhibited higher baseline AP amplitudes (˜70 mV) as compared to the second group (30-50 mV). In both groups, there appeared to be a small (if any) effect of the EMWs on the AP amplitude even at the highest IDP levels.

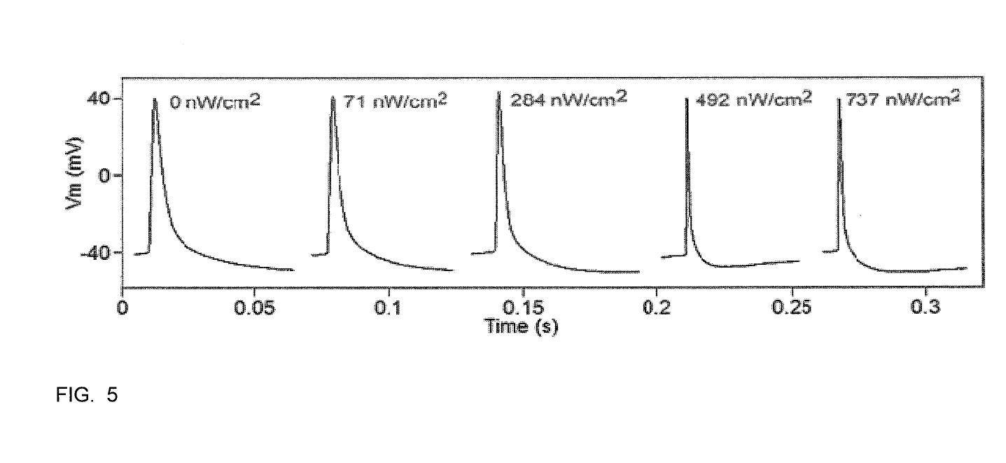

To examine the possible changes in the plasma membrane characteristics, the shape of the individual APs and the input resistance of the membrane were examined. The profiles of APs were compared prior to EMW exposure and at 50 s into the EMW exposure at several IPD levels (FIG. 5). Noticeable narrowing of the peaks can be readily observed at higher IPD levels. This narrowing was completely reversed to the baseline level within two minutes after the exposure (data not shown).

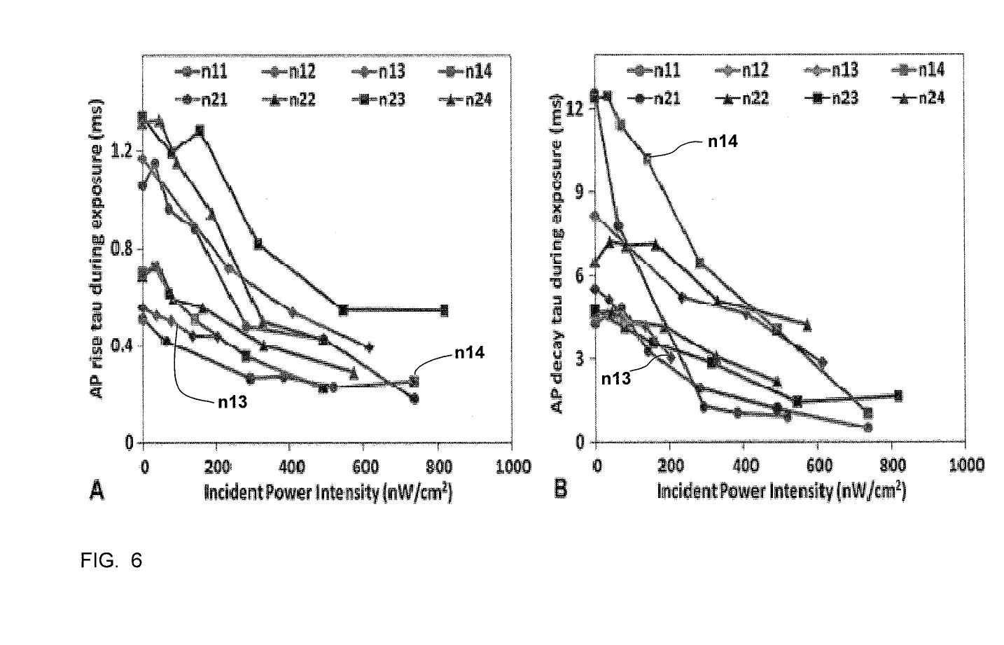

Quantification of the time constants for the AP rise and decay phases (τᵣᵢₛₑ and τ_{decay}) confirmed the remarkable decrease in both parameters proportional with the increasing strength of IPD and this effect was clearly dose-dependent on the level of IDP (FIG. 6).

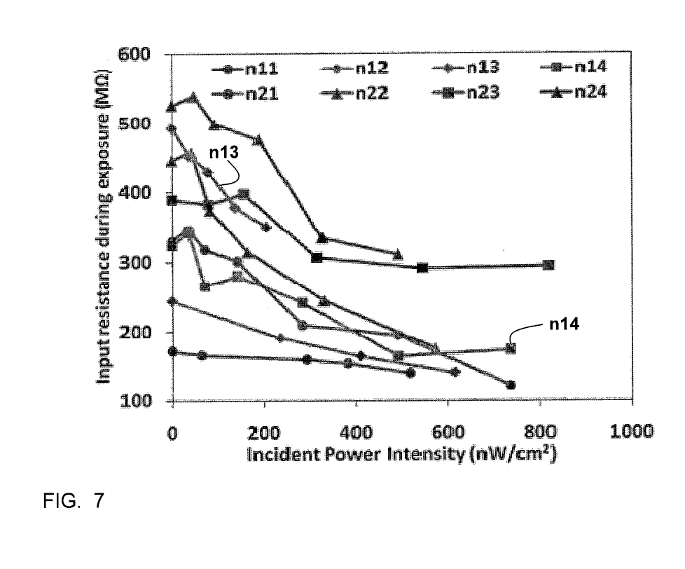

The membrane input resistance Rₙ was estimated as the change of the resting membrane potential during the injection of depolarizing current (ΔV/ΔI). Rₙ was strongly decreased during the EMW exposure. This effect, just as the peak narrowing, was also strongly IPD dose-dependent and in some neurons at high IPDs Rₙ value dropped below 200 MΩ (FIG. 7).

Out of the eight neurons studied, two neurons (n13 and n14) were examined in the absence of intracellular calcium signaling, accomplished by buffering the intracellular Ca2⁺ stores with 10 mM EGTA, added to the pipette solution. As evident from FIGS. 4, 6, and 7, there was no apparent difference in the responses of these neurons (n13 and n14) as compared to other neurons in this study. Their baseline values and EMW-induced changes in the τᵣᵢₛₑ, τ_{decay}, and Rₙ values were indistinguishable from the others, and their firing rates and AP amplitudes were close to those of the non-Ca2⁺-buffered neurons in group 1, n11 and n12.

Statistical evaluation of AP-related and membrane characteristics of two identified neuronal groups is presented in the Table 1. In group 1, EMW exposure reduced the firing rate to one third of the pre-exposure value, while in group 2 it was slightly increased. The AP amplitude in group 1 increased by 14%, while in group 2 it remained unchanged. Both groups of neurons exhibited similar shortening of the rise and decay times of the AP peak and similar reduction of the Rₙ.

TABLE 1
Analysis of the AP-related and membrane parameters in two subsets of cortical neurons.

| Neuronal | | Pre-MMW | During MMW exposure at following IDP ranges (nW/cm²) | | | | | |
|---|---|---|---|---|---|---|---|---|
| Group | Parameter | exposure | 30-50 | 70-90 | 140-200 | 250-330 | 490-600 | 700-800 |
| #1 | AP amp. (mW) | 72 ± 2 | 73 ± 1 | 74 ± 2 | 75 ± 2 | 76 ± 4 | 76 ± 5 | 82 ± 5* |
| (n = 4) | Firing rate (Hz) | 3.2 ± 0.2 | 2.8 ± 0.3 | 3.2 ± 0.3 | 3.1 ± 0.4 | 2.0 ± 0.6 | 0.9 ± 0.2* | 1.3 ± 0.4* |
| | τᵣᵢₛₑ (ms) | 0.73 ± 0.15 | 0.63 ± 0.1 | 0.51 ± 0.06 | 0.56 ± 0.08 | 0.4 ± 0.06 | 0.28 ± 0.06* | 0.25 ± 0.06* |
| | τ_{decay} (ms) | 9.7 ± 1.7 | 8.8 ± 3.7 | 7.9 ± 2.0 | 6.5 ± 1.9 | 3.8 ± 1.1 | 2.6 ± 0.9* | 1.0 ± 1.1* |
| | Rₙ (MΩ) | 308 ± 69 | 397 ± 56 | 287 ± 77 | 283 ± 54 | 229 ± 45 | 148 ± 18* | 173 ± 34* |
| #2 | AP amp. (mW) | 42 ± 4 | 41 ± 5 | 43 ± 6 | 45 ± 5 | 52 ± 6 | 50 ± 8 | 45 ± 13 |
| (n = 4) | Firing rate (Hz) | 5.6 ± 0.3 | 5.9 ± 0.1 | 6.0 ± 0.3 | 6.4 ± 0.4 | 7.1 ± 0.5 | 8.1 ± 1.1* | 6.5 ± 0.5 |
| | τᵣᵢₛₑ (ms) | 1.1 ± 0.15 | 1.07 ± 0.18 | 0.98 ± 0.14 | 0.92 ± 0.15 | 0.55 ± 0.09 | 0.42 ± 0.05** | 0.36 ± 0.18* |
| | τ_{decay} (ms) | 5.1 ± 0.5 | 5.5 ± 0.9 | 5.1 ± 0.7 | 4.5 ± 0.9 | 3.2 ± 0.7 | 2.3 ± 0.7* | 1.1 ± 0.6** |
| | Rₙ (MΩ) | 423 ± 42 | 448 ± 56 | 394 ± 38 | 372 ± 41 | 274 ± 29 | 243 ± 34* | 208 ± 86* |

Statistical significance (p) indicates difference from the pre-exposure control, based on the Dunnett post-hoc test, and is marked as follows: <0.05 (*), <0.01 (**).

The neuronal changes during exposure at the two strongest IPD levels were compared with changes, observed during heating of the ACSF bath with an in-line fluid heater, as reported previously by others {15}. While the temperature rise of ACSF during EMW exposure at the two highest IPD levels was only 1.8 and 2.7° C. versus 10° C. during bath heating, the observed EMW-induced decreases in the τᵣᵢₛₑ, τ_{decay}, and Rₙ were considerably more pronounced (Table 2). Specifically, the EMW exposure caused an 83% decrease in the AP width and 46% decrease in Rₙ, while the 10° C. bath heating produced only a 41% decrease in the AP width and 35% decrease in Rₙ.

TABLE 2
Analysis of the AP-related and membrane parameters of cortical neurons during
EMW exposure and bath heating. Statistical significance (p) indicates difference
from the pre-exposure control, based on the Dunnett post-hoc test, and is marked as
follows: <0.05 (*), <0.01 (**). Neuronal parameters for bath heating by an in-line
heater are adopted from {15}.

| | Pre-MMW | During exposure | During exposure | Pre-bath | During bath |
|---|---|---|---|---|---|
| Parameter | exposure | at 490-600 nW/cm² | at 700-800 nW/cm² | heating^{#} | heating^{#} |
| Bath T. (° C.) | 20.0 | 21.8 | 22.7 | 23 | 33 |
| AP amp. (mW) | 57 ± 6 | 61 ± 7 | 57 ± 12 | 90 ± 11 | 76 ± 8 |
| Firing rate (Hz) | 4.4 ± 0.5 | 5.0 ± 1.6 | 4.8 ± 1.8 | N.A. | N.A. |
| τᵣᵢₛₑ (ms) | 0.92 ± 0.12 | 0.36 ± 0.04 | 0.33 ± 0.11 | 0.90 ± 0.27 | 0.51 ± 0.13* |
| τ_{decay} (ms) | 7.4 ± 1.2 | 2.4 ± 0.5 | 1.1 ± 0.3 | 2.8 ± 1.1 | 1.7 ± 0.4* |
| Rₙ (MΩ) | 366 ± 43 | 202 ± 27 | 196 ± 51 | 389 ± 151 | 254 ± 96* |

### Conclusions

This study provides the first real-time examination of ex vivo single neuronal activity during EMW exposure. The findings in this study are the EMW-induced effects on neuronal firing, the shape of the APs, and the Rn. These effects were observed at extremely low IPD levels (<1 μW/cm²) of EMW and are stronger than those induced by 10° C. bath heating.

The observed EMW effects on the neuronal firing might be neuron subtype-specific. A subset of layer 2/3 cortical neurons with low firing rate (group 1) has been most susceptible to inhibition by the applied EMWs, as their firing was reduced to one third of the pre-exposure level. This dichotomy of responses might indicate a presence of different subtypes of pyramidal cells. Further studies with blockage of synaptic activity might be able to clarify the existence of such pyramidal neuron subpopulations and the mechanisms of their EMW-induced inhibition.

While the AP amplitude was not significantly affected by the EMW exposure, other AP parameters, τrise and τdecay were strongly affected by the EMWs, with the total AP duration reduced from 8.3 to 1.4 ms (83% decrease) for the highest IPD level. A key characteristic of the membrane conductivity, the Rn, was similarly dose-dependently reduced by increasing IPD levels, indicating the channel opening. The coincident decrease in Rn, τrise and τdecay is not surprising, as the narrowing of AP spikes is strongly correlated with decreased Rn {16}. The 10° C. bath heating of cortical neurons has a negligible effect on the AP amplitude and significantly reduces Rn, τrise and τdecay {15}. If EMW action on the neurons is largely thermal, then some of the same mechanisms implicated in the effects of heating might be involved. One such proposed mechanism for the heating-dependent changes in the neuronal membrane properties involves a temperature-dependent increase in K+ channel function (presumably due to faster channel activation), while Na+ channel function remains relatively constant {17; 18}. A modeling study of the Rn in the cortical pyramidal neurons also indicated an important role of the increased membrane conductance in the thermally-induced effects {16}. The EMW-induced effects in this study are considerably stronger than those evoked by 10° C. bath heating while the temperature rise at two highest IPD levels reached only 1.8 and 2.7° C. Potential temperature-independent mechanisms of EMW interaction with specific plasma membrane ion channels can be explored.

To evaluate possible EMW-specific mechanisms, the intracellular Ca²⁺-dependent second-messenger signaling in two neurons was blocked by using a pipette solution with a calcium chelator (10 mM EGTA), which buffers the intracellular calcium stores and thus eliminates the intracellular calcium-dependent fluctuations in the membrane potential {19; 20} This allowed for dissociation of a potential contribution of direct voltage-sensitive channel opening from indirect second-messenger mediated channel opening. The obtained results suggest that Ca²⁺-dependent second-messenger signaling does not significantly affect the induced changes in the AP generation and plasma membrane properties, leaving the direct voltage-sensitive channel opening as the main possible mechanism.

Thus, very low levels (10-1000× below recommended safe exposure limits) of EMW power can control (by inhibiting or exciting) the rate of action potential firing in the individually probed cortical neurons. By extension, EMWs are thought to inhibit or enhance neuronal activity in vivo via skin, catheter or through-tissue stimulation. Neuronal activity is a critical part of almost all biofunction. In particular, pain receptors in the skin are thought to be controlled with this technique.

### Example 2

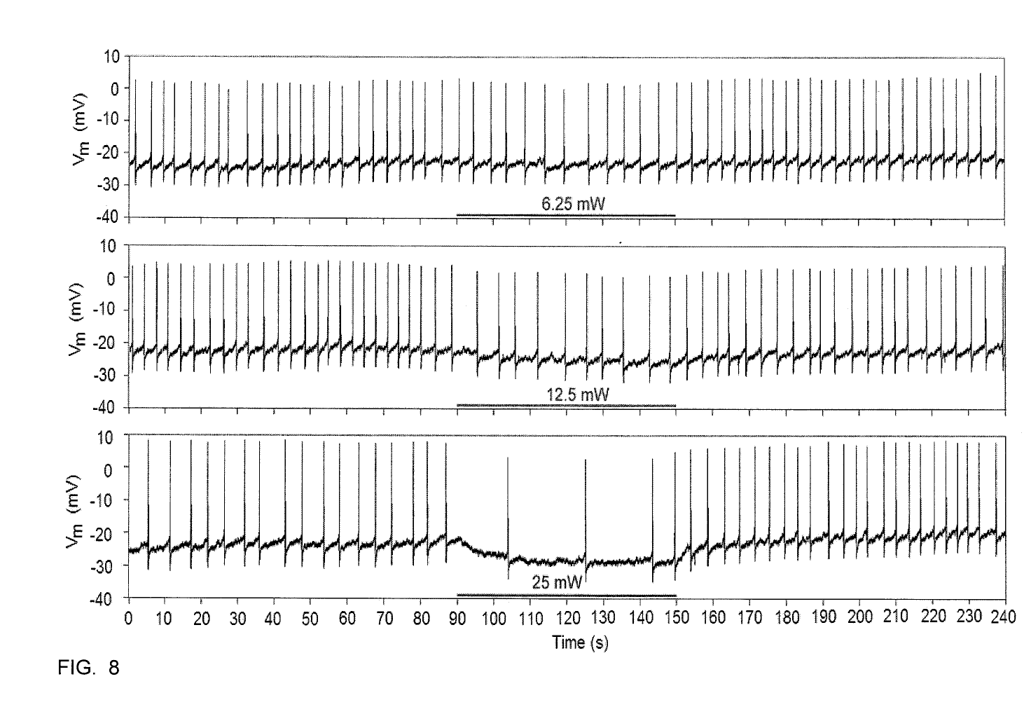

In other experiments {21}, one of the somatic ganglia of the common medicinal leech (Hirudo medicinalis) was surgically removed and placed in leech saline solution (NaCl, KC1, CaCl₂, MgCl₂, D-glucose, HEPES) in a polystyrene dish coated on the inside with 3 mm of melted paraffin. The ganglion was pinned onto the paraffin, and EMW power was applied from the bottom through the polystyrene and paraffin (both low loss materials in the EMW range). The large Retzius cells of the ganglion were patched with a pipette filled with 3M potassium acetate. Using an Axoclamp 2A intracellular recording device (Molecular Devices, Sunnyvale, Calif.), stimulation current was injected into the neuron to induce AP firing. The EMW power at 60 GHz was applied for 60 seconds. As with the cortical slices, the neuronal firing rate was impacted by the EMW exposure and was seen to decrease with increasing power (FIG. 8). The exact IPD was not known for this experiment but it was calculated to be below the MPE upon reaching the cell.

### Example 3

In a clinical setting, a person suspected of having a trauma, stroke, or neurodegenerative disorder, is equipped with a headcap with 64 EEG electrodes for recording neural activity over the cerebral cortex. Embedded into the headcap are 64 waveguides with planar horns that can be individually connected to a microwave source operating at 10 GHz. The baseline neural activity is recorded from EEG electrodes, then each of the waveguides is connected to the 10 GHz source, and changes in the EEG are recorded. If the cortical area under the planar horn is normal, the nearby EEG electrodes detect short-term changes in the local cortical excitability. However, if the function in the cortical area is altered due to a trauma, stroke, or neurodegenerative disorder, it will be either less responsive or more responsive to the applied EMW stimulation, depending on the nature of the neurological disorder.

### Example 4

In a clinical setting, a person who suffered a head concussion (for example in a car accident) several hours before is treated to suppress secondary brain trauma. Cortical neurons, overstimulated by the initial trauma, remain in the state of enhanced excitability leading to their additional damage (so called “excitotoxicity”). In order to inhibit the neuronal activity in the contused region of the cortex, a horn antenna is placed on the scalp immediately above the affected region. The horn is connected via waveguide to a microwave source operating at 5 GHz. Pulsatile stimulation is delivered with a pulse frequency of 5 Hz to induce short-term depression of neuronal excitability to rescue the neurons in the affected cortical region. Similar local inhibition of cortical excitability can be achieved with epidural electrical stimulation {22}, but the EMW approach will allow similar localization of stimulation without the need for invasive surgery to implant the electrode leads.

### Example 5

In a clinical setting, the EMW beam is used for intraretinal drug delivery to promote the survival of retinal neurons. The pupils are dilated with a single drop of tropicamide (1%) and phenylephrine (2.5%). The intraocular silicone oil tamponade (i.e. replacement of the vitreous fluid with heavier silicone oil) is performed to promote the retinal re-attachment. Fortuitously, the silicone oil provides much improved penetration of EMWs as compared to water. Just prior to the EMW exposure, the neuroprotective (e.g. lamotrigine) and anti-apoptotic drugs (e.g. X-linked inhibitor of apoptosis protein) are injected either intravenously or intraarterially. Then, pulses of EMWs at 30 GHz are applied toward the eye using a horn and a focusing EMW lens so that the focal depth is at the choroid (the connective tissue layer with blood vessels located between the retina and sclera). Localized choroidal heating induces a transient disruption in the blood-retinal barrier, allowing the drugs to be released into the retina and perform their therapeutic action. Detailed methods regarding the intraocular silicone oil tamponade following retinal detachment and action of neuroprotective and anti-apoptotic drugs have been described {23; 24}.

### Example 6

A person is placed in front of the computer monitor to observe a set of images to be memorized. Two EMW delivery sources are coupled to horns with a focusing movable lens and mounted on the scalp above the left and right anterior temporal lobes. Different duty cycles and/or pulsing frequencies are applied using the base EMW frequency of 10 GHz. The parameters of EMW irradiation are optimized to achieve reduced excitability in the left anterior temporal lobe and enhanced excitability in the left anterior temporal lobe. The EMW irradiation is applied during the memorization of images. Several minutes or hours later, the test is performed to evaluate how many of the images have been successfully memorized. As compared to the memorization without EMW exposure, the same person exposed to the EMWs during memorization will be able to recall the images with significantly improved accuracy. Detailed methods regarding the evaluation of visual memory during cortical electrical stimulation have been described {25}.

### REFERENCES

The following publications are incorporated by reference herein.
1 U.S. Pat. No. 5,716,377 (1998). Method of treating movement disorders by brain stimulation; Inventor: Rise, M. T., & King, G. W.; Assignee: Medtronic, Inc. (Minneapolis, Minn.).
2 U.S. Pat. No. 5,683,422 (1997). Method and apparatus for treating neurodegenerative disorders by electrical brain stimulation; Inventor: Rise, M. T.; Assignee: Medtronic, Inc. (Minneapolis, Minn.).
3 U.S. Pat. No. 5,752,979 (1998). Method of controlling epilepsy by brain stimulation; Inventor: Benabid, A. L.; Assignee: Medtronic, Inc. (Minneapolis, Minn.).
4 U.S. Pat. No. 7,013,177 (2006). Treatment of pain by brain stimulation; Inventor: Whitehurst, T. K., Woods, C. M., & Meadows, P. M.; Assignee: Advanced Bionics Corporation (Valencia, Calif.).
5 U.S. Pat. No. 5,501,703 (1996). Multichannel apparatus for epidural spinal cord stimulator; Inventor: Holsheimer, J., & Struijk, J. J.; Assignee: Medtronic, Inc. (Minneapolis, Minn.).
6 U.S. Pat. No. 5,330,515 (1994). Treatment of pain by vagal afferent stimulation; Inventor: Rutecki, P., Wernicke, J. F., & Terry Jr., R. S.; Assignee: Cyberonics, Inc.
7 U.S. Pat. No. 5,188,104 (1993). Treatment of eating disorders by nerve stimulation; Inventor: Wernicke, J. F., Terry Jr., R. S., & Baker Jr., R. G.; Assignee: Cyberonics, Inc. (Webster, Tex.).
8 U.S. Pat. No. 5,299,569 (1994). Treatment of neuropsychiatric disorders by nerve stimulation; Inventor: Wernicke, J. F., Terry Jr., R. S., & Zabara, J.; Assignee: Cyberonics, Inc. (Webster, Tex.).
9 Pepe, D., & Zito, D. (2010, 26-28 Apr. 2010). 60-GHz transceivers for wireless HD uncompressed video communication in nano-era CMOS technology. Paper presented at the MELECON 2010-2010 15th IEEE Mediterranean Electrotechnical Conference.
10 Sato, H., Sawaya, K., Mizuno, K., Uemura, J., Takeda, M., Takahashi, J., et al. (2009, 25-28 Oct. 2009). Development of 77 GHz millimeter wave passive imaging camera. Paper presented at the Sensors, 2009 IEEE.
11 Lynch, J. J., Macdonald, P. A., Moyer, H. P., & Nagele, R. G. (2010). Passive millimeter wave imaging sensors for commercial markets. Appl Opt, 49(19), E7-12.
12 Pikov, V., Arakaki, X., Harrington, M., Fraser, S. E., & Siegel, P. H. (2010). Modulation of neuronal activity and plasma membrane properties with low-power millimeter waves in organotypic cortical slices. J Neural Eng, 7, 045003.
13 Siegel, P. H., & Pikov, V. (2010). THz in biology and medicine: toward quantifying and understanding the interaction of millimeter- and submillimeter-waves with cells and cell processes. SPIE BiOS, 7562, 75620H.
14 Chou, C.-K., & D'Andrea, J. (Eds.). (2005). IEEE Standard for Safety Levels With Respect to Human Exposure to Radio Frequency Electromagnetic Fields, 3 kHz to 300 GHz.
15 Lee, J. C. F., Callaway, J. C., & Foehring, R. C. (2005). Effects of Temperature on Calcium Transients and Ca2+-Dependent After hyperpolarizations in Neocortical Pyramidal Neurons. J Neurophysiol, 93(4), 2012-2020.
16 Trevelyan, A. J., & Jack, J. (2002). Detailed passive cable models of layer 2/3 pyramidal cells in rat visual cortex at different temperatures. J Physiol, 539(2), 623-636.
17 Volgushev, M., Vidyasagar, T. R., Chistiakova, M., Yousef, T., & Eysel, U. T. (2000). Membrane properties and spike generation in rat visual cortical cells during reversible cooling. J Physiol, 522 Pt 1, 59-76.
18 Cao, X.-J., & Oertel, D. (2005). Temperature Affects Voltage-Sensitive Conductances Differentially in Octopus Cells of the Mammalian Cochlear Nucleus. J Neurophysiol, 94(1), 821-832.
19 Harks, E. G. A., Tones, J. J., Cornelisse, L. N., Ypey, D. L., & Theuvenet, A. P. R. (2003). Ionic basis for excitability of normal rat kidney (NRK) fibroblasts. J Cell Physiol, 196(3), 493-503.
20 Maravall, M., Mainen, Z. F., Sabatini, B. L., & Svoboda, K. (2000). Estimating Intracellular Calcium Concentrations and Buffering without Wavelength Ratioing. Biophys J, 78(5), 2655-2667.
21 Pikov, V., & Siegel, P. H. (2011). Photons and Neurons: Monitoring millimeter wave-induced changes in neuronal activity using the leech ganglion. SHE BiOS, (in press).
22 Pikov, V., & McCreery, D. B. (2009). Spinal Hyperexcitability and Bladder Hyperreflexia during Reversible Frontal Cortical Inactivation Induced by Low-Frequency Electrical Stimulation in the Cat. J Neurotrauma, 26(1), 109-119.
23 Guizzo, R., Paques, M. W., Anhezini, L., Simon, C. R., Scott, I. U., Jorge, R., et al. (2008). Neuroprotective Effects of Oral Lamotrigine Administration on Rabbit Retinas After Pars Plana Vitrectomy and Silicone Oil Injection. Retina, 28(4), 638-644.
24 Zadro-Lamoureux, L. A., Zacks, D. N., Baker, A. N., Zheng, Q.-D., Hauswirth, W. W., & Tsilfidis, C. (2009). Effects on XIAP Retinal Detachment-Induced Photoreceptor Apoptosis. Investigative Ophthalmology & Visual Science, 50(3), 1448-1453.
25 Chi, R. P., Fregni, F., & Snyder, A. W. (2010). Visual memory improved by non-invasive brain stimulation. Brain Research, 1353, 168-175.

Although the present invention has been described in connection with the preferred embodiments, it is to be understood that modifications and variations may be utilized without departing from the principles and scope of the invention, as those skilled in the art will readily understand. Accordingly, such modifications may be practiced within the scope of the invention and the following claims.

What is claimed is:

1. A method of diagnosing a neural disorder, comprising:
applying electromagnetic waves (EMWs) at a frequency in the range of 3 to 300 GHz to a neural target of a subject so as to alter a neural property of the target,
comparing the neural property of the target after EMW application to the same neural property of a similarly treated control target, and
diagnosing a neural disorder in the subject when a difference in the neural properties is observed.

2. The method of claim 1, further comprising discontinuing the EMW application in the event that the EMWs are applied in a manner that exceeds a predetermined upper EMW application limit.

3. The method of claim 1, wherein the applying of the EMWs comprises applying several EMW amplitudes or applying in a duty cycled manner.

4. The method of claim 1, wherein the neural target is selected from the group consisting of cerebral cortex, deep brain, brainstem, spinal cord, optic tract, optic nerve, retina, cranial nerves, spinal roots, peripheral nerves, and nerve endings in the skin, and a combination thereof.

5. A method of treating a neural disorder, comprising:
exposing a neural target of a subject in need of such treatment to electromagnetic waves (EMWs) at a frequency in the range of 3 to 300 GHz in an amount sufficient to alter a neural property of the neural target, thereby conferring a therapeutic benefit on the subject, wherein the neural target is identified with a neural disorder.

6. The method of claim 5, further comprising discontinuing the EMW exposure in the event that the EMWs are applied in a manner that exceeds a predetermined upper EMW exposure limit.

7. The method of claim 5, wherein the exposing of the neural target comprises exposing the neural target to several EMW amplitudes or to EMWs in a duty cycled manner.

8. The method of claim 5, further comprising monitoring neuronal function of the subject.

9. The method of claim 5, wherein the neural target is selected from the group consisting of cerebral cortex, deep brain, brainstem, spinal cord, optic tract, optic nerve, retina, cranial nerves, spinal roots, peripheral nerves, and nerve endings in the skin, and a combination thereof.

10. A method of delivering a drug, comprising:
exposing a neural target in a subject's central nervous system or optic system to electromagnetic waves (EMWs) at a frequency in the range of 3 to 300 GHz in an amount sufficient to alter the subject's blood-brain or blood-retinal barrier, respectively, and
administering a drug to the subject before, during, or after the EMW exposure, or any combination thereof, such that the drug crosses the blood-brain or blood-retinal barrier.

11. A method of cognitive enhancement, comprising
exposing a neural target in a subject's central nervous system to electromagnetic waves (EMWs) at a frequency in the range of 3 to 300 GHz in an amount sufficient to alter a neural property of the neural target, thereby increasing cognitive ability in the subject.

12. An electromagnetic wave (EMW) stimulation system for a subject, comprising:
a housing that is implantable, portable, head-mountable, skin-mountable, or eye-mountable, or a combination thereof;
a control unit, for controlling production of EMWs;
at least one EMW source carried by the housing and operatively coupled to the control unit; and
an EMW delivery device comprising at least one emission site operatively coupled to the EMW source.

13. The system of claim 12, wherein the housing is constructed from flexible polymer substrate upon which the wave delivery device is positioned, the wave delivery device comprising metal traces for forming a surface waveguide and an output EMW shaping element coupled to the waveguide.

14. The system of claim 12, wherein the EMW delivery device comprises an elongate member and first and second EMW emission sites carried by the elongate member, wherein the first and second emission sites are separated along a length of the elongate member by a predetermined distance.

15. The system of claim 12, wherein the EMW delivery device comprises:
an elongate member that comprises a proximal segment and a distal segment, where the segments have different geometric shapes; and
a first wave emission site carried by the elongate member.

16. The system of claim 12, further comprising a unit for monitoring neuronal function operatively coupled to the control unit.

17. The system of claim 12, wherein the control unit is carried by the housing.
